(19) United States
(12) Patent Application Publication
(10) Pub. No.: US 2021/0353614 A1
(43) Pub. Date: Nov. 18, 2021

(54) COMPOUNDS AND USES THEREOF TO TREAT TUMORS IN A SUBJECT

(71) Applicants: Merck Patent GMBH, Darmstadt (DE); Pfizer, Inc., New York, NY (US)

(72) Inventors: Astrid Zimmermann, Muehltal (DE); Maria Jesus Ortiz Ruiz, Frankfurt am Main (DE); Heike Dahmen, Darmstadt (DE); Thomas Grombacher, Reinheim (DE)

(21) Appl. No.: 16/980,058

(22) PCT Filed: Mar. 13, 2019
(86) PCT No.: PCT/EP2019/056287

§ 371 Date: Sep. 11, 2020

(30) Foreign Application Priority Data
Mar. 14, 2018 (EP) 18161849.7

(51) Int. Cl. A61K 31/4709; A61K 39/395; A61P 35/00; A61K 31/4745

(52) U.S. Cl. CPC A61K 31/4709; A61K 31/4745; A61P 35/00; A61K 39/3955

(57) ABSTRACT

The present invention relates to compounds that can be used to induce immunogenic cell death (ICD). In some instances, two or more compounds are combined to induce ICD. The invention also relates to the combination of ICD-inducing compounds with an agent stimulating the immune system, such as an immune checkpoint inhibitor, in particular, to 5 treat cancer or inhibit tumor growth.

### FIELD OF THE INVENTION

The present invention relates to compounds for inducing immunogenic cell death. The present invention also relates to combination therapies useful for the treatment of a tumor. In particular, the invention relates to the combined administration of a topoisomerase inhibitor, an ATM inhibitor and an agent stimulating the immune system. Compositions and kits comprising these compounds are also disclosed.

### BACKGROUND

Immunogenic cell death (ICD) is a form of cell death that can trigger an immune response. ICD is characterized by the release of danger signals from the dying cells, which are known as damage-associated molecular patterns (DAMPs). Important DAMPs include the exposure of CALR, PDIA3, HSP70 and HSP90 on the membrane surface and the release of HMGB1 and ATP from the dying cells. These DAMPs then trigger an immune response, e.g., through interaction with pattern recognition receptors (PRRs) on antigen-presenting cells (APCs).

It has been observed that a subset of cancer treatments, such as oxaliplatin and radiotherapy, can trigger ICD of cancer cells and ICD is understood to contribute to the anti-tumor effect of the therapy. Importantly, ICD stimulates the immune system to attack the cancer and thus provides a long-lasting protection against cancer recurrence and metastasis. There remains a need to identify further agents that can promote ICD.

### SUMMARY OF THE INVENTION

Provided herein are methods for inducing ICD, e.g. for treating a tumor, preferably a cancer, comprising administering to the subject a topoisomerase inhibitor.

Optionally, the topoisomerase inhibitor is administered with an ATM (ataxia-telangiectasia mutated) inhibitor and/or an agent stimulating the immune system.

Also provided herein are methods for treating a tumor, preferably a cancer, comprising administering to the subject a topoisomerase inhibitor, an ATM inhibitor and an agent stimulating the immune system.

Also provided herein are kits and compositions comprising a topoisomerase inhibitor, an ATM inhibitor and an agent stimulating the immune system.

### BRIEF DESCRIPTION OF THE FIGURES

FIG. 1 shows the combination effect of two ATM inhibitors with various chemotherapeutic agents, including the topoisomerase inhibitors irinotecan, topotecan, and etoposide on the cell growth of 35 cancer cell lines. The combination effect is expressed as BLISS excess over the additive monotherapy effects. Positive BLISS excess values are synergistic effects, and negative BLISS excess values are antagonistic effects. Values between −0.1 and 0.1 are considered close to the linear combination effect.

FIG. 2 shows the effect of two different ATM inhibitors on growth inhibition as single agent or in combination with different fixed concentrations of SN38 on two ATM proficient cell lines (HCT116 and SW620) and on two ATM deficient cell lines (SKCO1 and NCI-H23).

FIG. 3 shows the combination effect of two ATM inhibitors with SN38 in a human colon xenograft model implanted subcutaneously into mice. Tumor volume was followed over time and is blotted on the y-axis. Vehicle treated tumors are considered as a reference. The antitumor effect of the different treatment modalities and combinations can be judged by reduced tumor volume over time compared to the reference. 10 animals were used per treatment arm, the mean tumor volumes are blotted.

FIG. 4 shows the effect of the topoisomerase inhibitor SN38 as a single agent or in combination with an ATM inhibitor on immunogenic cell death induction and apoptosis as seen by A) the increased exposure of Calreticulin (CRT), HSP70 and HSP90, B) increase in ATP and HMGB1 release and C) activation of caspase-3/-7. Two biological repeats are shown for each experiment. Unstained (not shown) and appropriate isotype-matched antibodies were used as controls for FACS analyses.

### DETAILED DESCRIPTION OF THE INVENTION

The ability of agents stimulating the immune system, such as checkpoint inhibitors, to treat cancers is dependent on the existence of tumour antigen-specific T-cells within tumour tissue. This requires that tumour tissue expresses antigens that differentiate themselves from their non-transformed counterparts like for example, through novel protein products known as neoantigens. Tumor neoantigen burden strongly correlates with immunogenicity and with sensitivity to, e.g., checkpoint inhibitor therapies, implying that poorly immunogenic tumours should be largely resistant to these agents. Therapies that serve to liberate tumour antigen available for uptake by APCs, such as those inducing immunogenic cell death, are likely to promote effective anti-tumor immunity, especially when further combined with agents stimulating the immune system, such as checkpoint inhibitors.

The present inventors discovered that topoisomerase inhibitors can trigger ICD of tumor cells. Furthermore, various combinations of topoisomerase inhibitors and ATM inhibitors were observed to act at least additively or synergistically, e.g., in promoting ICD.

The present disclosure therefore provides a method for inducing ICD, e.g., of a tumor cell, preferably a cancer cell, comprising the administration of a topoisomerase inhibitor and, optionally, the additional administration of an ATM inhibitor and/or an agent stimulating the immune system.

The present disclosure also provides a method for treating a tumor comprising the administration of a topoisomerase inhibitor, an ATM inhibitor and an agent stimulating the immune system. In a preferred embodiment, the treated tumor is a cancerous or malignant tumor.

Administration of one treatment modality in addition to another treatment modality refers to administration of one treatment modality before, during, or after administration of the other treatment modality to the subject. This means with regard to administration of the topoisomerase inhibitor, the ATM inhibitor and the agent stimulating the immune system that each of the topoisomerase inhibitor, the ATM inhibitor and the agent stimulating the immune system are administered to the patient in any order (i.e., simultaneously or sequentially) or together in a single composition, formulation or unit dosage form. In some embodiments, the topoisomerase inhibitor, the ATM inhibitor and the agent stimulating the immune system are administered simultaneously or sequentially. In certain embodiments, the topoisomerase inhibitor, the ATM inhibitor and the agent stimulating the immune system are administered simultaneously in the same composition comprising the topoisomerase inhibitor, the ATM inhibitor and the agent stimulating the immune system. In certain embodiments, the topoisomerase inhibitor, the ATM inhibitor and the agent stimulating the immune system are administered simultaneously in separate compositions, i.e., wherein the topoisomerase inhibitor, the ATM inhibitor and the agent stimulating the immune system are administered simultaneously each in a separate unit dosage form. It will be appreciated that the topoisomerase inhibitor, the ATM inhibitor and the agent stimulating the immune system can be administered on the same day or on different days and in any order as according to an appropriate dosing protocol.

If there is one treatment modality administered in addition to another treatment modality, the treatment modalities are administered within the same treatment regimen. For instance, they are used together as first-, second- or third-line treatment.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

Preferably, the “subject” is a human. Generally, the subject is a human diagnosed or at risk for suffering from one or more symptoms of a tumor or cancer. In certain embodiments a “subject” may refer to a non-human mammal, such as a non-human primate, a dog, cat, rabbit, pig, mouse, or rat, or animals used in screening, characterizing, and evaluating drugs and therapies.

Specific types of tumor or cancer to be treated according to the invention include, but are not limited to, tumor or cancer selected from the group consisting of colorectal, breast, ovarian, pancreatic, gastric, prostate, renal, cervical, myeloma, lymphoma, leukemia, thyroid, endometrial, uterine, bladder, neuroendocrine, head and neck, liver, nasopharyngeal, testicular, small cell lung cancer, nonsmall cell lung cancer, melanoma, basal cell skin cancer, squamous cell skin cancer, dermatofibrosarcoma protuberans, Merkel cell carcinoma, glioblastoma, glioma, sarcoma, mesothelioma, and myelodisplastic syndromes. Preferably, the tumor or cancer to be treated expresses ATM.

In some embodiments, the topoisomerase inhibitor inhibits topoisomerase I and/or II. Topoisomerase inhibitors are, for example, topotecan, hycaptamine, irinotecan, rubitecan, 6-ethoxypropionyl-3′,4′-O-exobenzylidenechartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methyl-1H,12H-benzo[de]pyrano[3′,4′:b,7]indolizino[1,2b]quinoline-10,13(9H,15H)-dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxyetoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydroxy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)-ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one and dimesna.

Preferably, the topoisomerase inhibitor is a camptothecin derivative, preferably any one of irinotecan, topotecan or etoposide, more preferably irinotecan. Irinotecan is also known as CPT-11 and has the following structure:

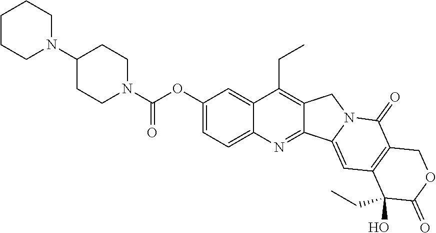 [SMILES: CCc1c2c(nc3ccc(OC(=O)N4CCC(N5CCCCC5)CC4)cc13)-c1cc3c(c(=O)n1C2)COC(=O)[C@]3(O)CC]

Unless otherwise specified, the term “irinotecan” also includes pharmaceutically acceptable salts thereof, e.g., the hydrochloride salt, and metabolites thereof, such as SN-38.

Preferably, Irinotecan is administered to the patient in an amount of 20 mg to 300 mg, more preferably 40 to 200 mg, within a time period of 2 to 4 weeks and preferably within a time period of about three weeks, which time periods are preferably to be regarded as one cycle. More preferably, the amount of Irinotecan administered to the patient is given in mg per square metre of the by the surface of the patient, i.e. in mg/m². Accordingly, more preferably the Irinotecan is administered to the patient in an amount of 30 mg/m² to 100 mg/m², more preferably 50 mg/m² to 70 mg/m², for example in an amount of about 60 mg/m², within a time period of 2 to 4 weeks and preferably within a time period of about three weeks, which time periods are preferably to be regarded as one cycle. Even more preferably, the amount of Irinotecan to be administered to the patient is administered to the patient on one day, preferably at the beginning of one cycle with respect to the Irinotecan. Especially preferably, the Irinotecan is administered to the patient in an amount of about 40 mg/m² to 60 mg/m² per day on days 1 of a cycle consisting of about 21 days. Preferably, 2 to 12 cycles, more preferably 4 to 8 cycles and especially about 6 cycles are applied to the patient with respect to Irinotecan, preferably substantially without a pause. The whole procedure/regimen described above with respect to the Irinotecan can be repeated one or more times, preferably one to 12 times and especially 2 to 6 times, for example about 5 times, preferably with a pause in between each repetition of the procedure/regimen.

Preferably, the ATM inhibitor has an IC₅₀ below 1 mM, more preferably below 100 μM, more preferably below 1 μM, more preferably below 100 nM and most preferably below 10 nM. Preferably, the ATM inhibitor possesses a specificity for inhibiting ATM over other kinases, preferably ATR, of at least 10-fold, more preferably at least 100-fold, most preferably at least 1000-fold, as measured by the ratio of IC₅₀ for ATM over IC₅₀ for other kinases.

Assays for measuring the IC₅₀ of an ATM inhibitor are well known to the person skilled in the art. The IC₅₀ may, for instance, be determined with the aid of the following biochemical ATM kinase assay. The assay consists of two steps: the enzymatic reaction and the detection step. Firstly, ATM protein and the test substance are incubated at different concentrations with addition of substrate protein p53 and ATP. ATM mediates the phosphorylation of p53 at several positions, including at amino acid S15. The amount of phosphorylated p53 is determined with the aid of specific antibodies and the TR-FRET technique. The enzymatic ATM assay is carried out as TR-FRET (HTRF™, Cisbio Bioassays) based 384-well assay. In the first step, purified human recombinant ATM (human ATM, full length, GenBank ID NM_000051, expressed in a mammal cell line) is incubated in assay buffer for 15 minutes with the ATM inhibitor in various concentrations and without test substance as negative or neutral control. The assay buffer comprises 25 mM HEPES pH 8.0, 10 mM Mg(CH₃COO)₂, 1 mM MnCl₂, 0.1% BSA and 0.01% Brij® 35, 5 mM dithiothreitol (DTT). The test-substance solutions are dispensed into the microtitre plates using an ECHO 555 (Labcyte). In the second step, purified human recombinant cmyc-labelled p53 (human p53, full length, GenBank ID BC003596, expressed in Sf21 insect cells) and ATP are added, and the reaction mixture is incubated at 22° C. for 30-35 minutes. The pharmacologically relevant assay volume is 5 μl. The final concentrations in the assay during incubation of the reaction mixture are 0.3-0.4 nM ATM, 50-75 nM p53 and 10 μM ATP. The enzymatic reaction is stopped by addition of EDTA. The formation of phosphorylated p53 as the result of the ATM-mediated reaction in the presence of ATP is detected via specific antibodies [labelled with the fluorophorene europium (Eu) as donor and d2 as acceptor (Cisbio Bioassays)] which enable FRET. 2 μl of antibody-containing stop solution (12.5 mM HEPES pH 8.0, 125 mM EDTA, 30 mM sodium chloride, 300 mM potassium fluoride, 0.1006% Tween-20, 0.005% Brij® 35, 0.21 nM anti-phospho-p53(ser15)-Eu antibody and 15 nM anti-cmyc-d2 antibody) are added to the reaction mixture. After incubation, usually for 2 hours (between 1.5 and 15 h), for signal development, the plates are analysed in a plate reader (EnVision, PerkinElmer) using TRF mode (and with laser excitation). After excitation of the donor europium at a wavelength of 340 nm, the emitted fluorescence light both of the acceptor d2 at 665 nm and also of the donor Eu at 615 nm is measured. The amount of phosphorylated p53 is directly proportional to the quotient of the amounts of light emitted, i.e. the relative fluorescence units (RFU) at 665 nm and 615 nm. The measurement data are processed by means of Genedata Screener software. IC₅₀ determinations are carried out, in particular, by fitting a dose/action curve to the data points by means of nonlinear regression analysis.
IC₅₀=half-maximum inhibitory concentration
ATP=adenosine triphosphate
TR-FRET=time-resolved fluorescence resonance energy transfer
HTRF®=homogeneous time resolved fluorescence
HEPES=2-(4-(2-hydroxyethyl)-1-piperazinyl)ethanesulfonic acid
Mg(CH₃COO)₂=magnesium acetate
MnCl₂=manganese(II) chloride
BSA=bovine serum albumin
EDTA=ethylenediamine tetraacetate
TRF=time resolved fluorescence

The ATM inhibitor may be selected from the following group:

| Compound | CAS-No.: | Chemical structure |
|---|---|---|
| KU-55933 | 587871-26-9 | 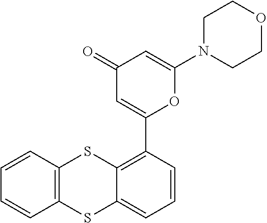 [SMILES: O=c1cc(-c2cccc3c2Sc2ccccc2S3)oc(N2CCOCC2)c1] |
| KU-60019 | 925701-46-8 | 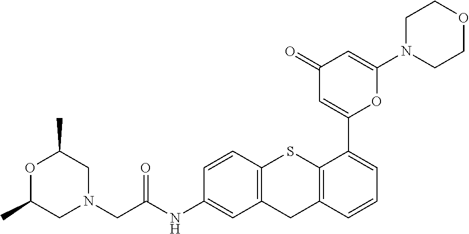 [SMILES: C[C@@H]1CN(CC(=O)Nc2ccc3c(c2)Cc2cccc(-c4cc(=O)cc(N5CCOCC5)o4)c2S3)C[C@H](C)O1] |
| Wortmannin | 19545-26-7 | 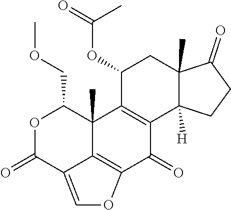 [SMILES: COC[C@H]1OC(=O)c2coc3c2[C@@]1(C)C1=C(C3=O)[C@@H]2CCC(=O)[C@@]2(C)C[C@H]1OC(C)=O] |
| CP-466722 | 1080622-86-1 | 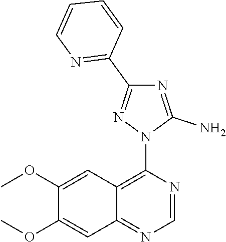 [SMILES: COc1cc2ncnc(-n3nc(-c4ccccn4)nc3N)c2cc1OC] |
| Torin 2 | 1223001-51-1 | 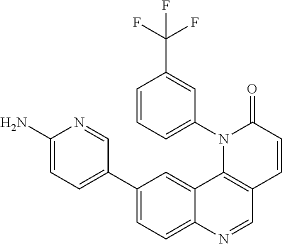 [SMILES: Nc1ccc(-c2ccc3ncc4ccc(=O)n(-c5cccc(C(F)(F)F)c5)c4c3c2)cn1] |
| CGK733 | 905973-89-9 | 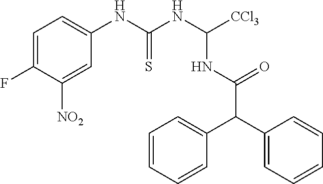 |
| ATM Inhibitor-1 | 2135639-94-8 | 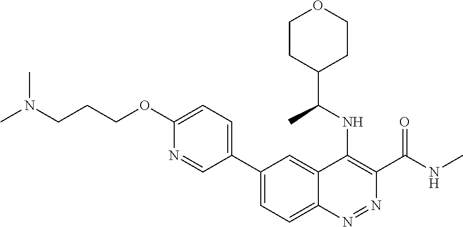 |
| AZD1390 | 2089288-03-7 | 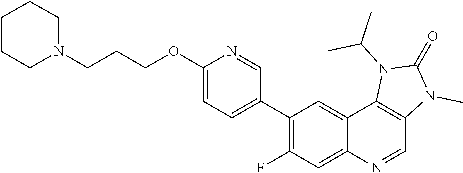 [SMILES: CC(C)n1c(=O)n(C)c2cnc3cc(F)c(-c4ccc(OCCCN5CCCCC5)nc4)cc3c21] |
| KU-59403 | 845932-30-1 | 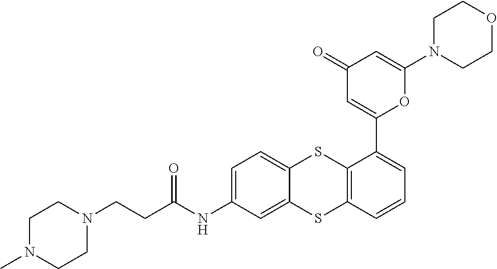 [SMILES: CN1CCN(CCC(=O)Nc2ccc3c(c2)Sc2cccc(-c4cc(=O)cc(N5CCOCC5)o4)c2S3)CC1] |
| AZD0156 | 1821428-35-6 | 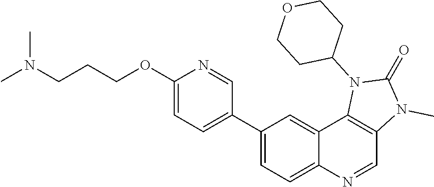 |

In some embodiments, the ATM inhibitor is an imidazo[4,5-c]quinoline derivative. More preferably, the ATM inhibitor is any one selected from the group consisting of the compounds of claim 6 of WO 2012/028233 A1 or the compounds of claim 18 of WO 2016/155884 A1. Most preferably, the ATM inhibitor is 3-Fluoro-4-[7-methoxy-3-methyl-8-(1-methyl-1H-pyrazol-4-yl)-2-oxo-2,3-dihydroimidazo[4,5-c]-quinolin-1-yl]benzonitrile (also referred to as Compound 1).

Compound 1 is described in detail in WO 2012/028233 A1, published on Mar. 8, 2012. Compound 1 is designated as compound 36 in Table 5 of the WO 2012/028233 A1 publication.

Unless otherwise specified, the term “Compound 1” also includes pharmaceutically acceptable salts thereof.

Possible dosages of Compound 1 are described in WO 2012/028233 A1.

In another preferred embodiment, the ATM inhibitor is 8-(1,3-Dimethyl-1H-pyrazol-4-yl)-1-(3-fluoro-5-methoxy-pyridin-4-yl)-7-methoxy-3-methyl-1,3-dihydro-imidazo[4,5-c]quinolin-2-one or a pharmaceutically acceptable salt thereof.

Preferably, the ATM inhibitor is Compound 1 or 8-(1,3-Dimethyl-1H-pyrazol-4-yl)-1-(3-fluoro-5-methoxy-pyridin-4-yl)-7-methoxy-3-methyl-1,3-dihydro-imidazo[4,5-c]quinolin-2-one or a pharmaceutically acceptable salt thereof.

The term “agent stimulating the immune system” refers to an agent capable of increasing the activity of the immune system. In some embodiments, the agent stimulating the immune system is an immune checkpoint inhibitor, such as an antagonist of the PD-1 (Programmed cell death 1) pathway (also referred to as a “PD-1 antagonist”). Preferably, the agent stimulating the immune system is an anti-PD-L1 (Programmed death-ligand 1) antibody.

“Anti-PD-L1 antibody” means an antibody that blocks binding of PD-L1 expressed on a cancer cell to PD-1. In any of the treatment method, medicaments and uses of the present invention in which a human subject is being treated, the anti-PD-L1 antibody specifically binds to human PD-L1 and blocks binding of human PD-L1 to human PD-1. The antibody may be a monoclonal antibody, human antibody, humanized antibody or chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen-binding fragment is selected from the group consisting of Fab, Fab′-SH, F(ab′)2, scFv and Fv fragments. Examples of antibodies that bind to human PD-L1, and useful in the treatment method, medicaments and uses of the present invention, are described in WO 2007/005874, WO 2010/036959, WO 2010/077634, WO 2010/089411, WO 2013/019906, WO 2013/079174, WO 2014/100079, WO 2015/061668, and U.S. Pat. Nos. 8,552,154, 8,779,108 and 8,383,796. Specific anti-human PD-L1 antibodies useful as the PD-L1 antibody in the treatment method, medicaments and uses of the present invention include, for example without limitation, avelumab (MSB0010718C), nivolumab (BMS-936558), MPDL3280A (an IgG1-engineered, anti-PD-L1 antibody), BMS-936559 (a fully human, anti-PD-L1, IgG4 monoclonal antibody), MED14736 (an engineered IgG1 kappa monoclonal antibody with triple mutations in the Fc domain to remove antibody-dependent, cell-mediated cytotoxic activity), an antibody which comprises the heavy chain and light chain variable regions of SEQ ID NO:24 and SEQ ID NO:25, respectively, of WO 2013/079174, and an antibody which comprises the light and heavy chain sequences of SEQ ID NO: 1 and SEQ ID NO: 3, respectively, of WO 2015/118175. In some preferred embodiments, the anti-PD-L1 antibody comprises the heavy chain sequence SEQ ID NO: 32 and the light chain sequence SEQ ID NO: 33 of WO 2013/079174. In some preferred embodiments, the anti-PD-L1 antibody is avelumab. In some embodiments, the anti-PD-L1 antibody is used in the treatment of a human subject. In some embodiments, PD-L1 is human PD-L1.

In some embodiments, the anti-PD-L1 antibody is administered intravenously (e.g., as an intravenous infusion) or subcutaneously. Preferably, the anti-PD-L1 antibody is administered as an intravenous infusion. More preferably, the inhibitor is administered for 50-80 minutes, most preferably as a one-hour intravenous infusion. In some embodiment, the anti-PD-L1 antibody is administered at a dose of about 10 mg/kg body weight every other week (i.e., every two weeks, or “Q2W”).

In some embodiments, any of the above methods of treatment is used in combination with a further chemotherapy (CT), radiotherapy (RT) or chemoradiotherapy (CRT).

The radiotherapy can be a treatment given with electrons, photons, protons, alfa-emitters, other ions, radio-nucleotides, boron capture neutrons and combinations thereof. In some embodiments, the radiotherapy comprises about 35-70 Gy/20-35 fractions.

“Chemotherapy” is a therapy involving a chemotherapeutic agent, which is a chemical compound useful in the treatment of tumor or cancer. Examples of chemotherapeutic agents include alkylating agents such as thiotepa and cyclophosphamide; alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethiylenethiophosphoramide, and trimethylolomelamine; acetogenins (especially bullatacin and bullatacinone); delta-9-tetrahydrocannabinol (dronabinol); beta-lapachone; lapachol; colchicines; betulinic acid; a camptothecin (including the synthetic analogue topotecan (CPT-11 (irinotecan), acetylcamptothecin, scopolectin, and 9-aminocamptothecin); bryostatin; pemetrexed; callystatin; CC-1065 (including its adozelesin, carzelesin, and bizelesin synthetic analogues); podophyllotoxin; podophyllinic acid; teniposide; cryptophycins (particularly, cryptophycin 1 and cryptophycin 8); dolastatin; duocarmycin (including the synthetic analogues KW-2189 and CB1-TM1); eleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Nicolaou et al. (1994) Angew. Chem Intl. Ed. Engl. 33: 183); dynemicin including dynemicin A; an esperamicin; as well as neocarzinostatin chromophore and related chromoprotein enediyne antibiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, doxorubicin HCl liposome injection, and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites such as methotrexate, gemcitabine, tegafur, capecitabine, an epothilone, and 5-fluorouracil (5-FU); folic acid analogues such as denopterin, methotrexate, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, floxuridine, and imatinib (a 2-phenylaminopyrimidine derivative), as well as other c-Kit inhibitors; anti-adrenals such as aminoglutethimide, mitotane, and trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially, T-2 toxin, verracurin A, roridin A, and anguidine); urethan; vindesine; dacarbazine; mannomustine; mitobronitol; mitolactol; pipobroman; gacytosine; arabinoside (“Ara-C”); thiotepa; taxoids, e.g., paclitaxel, albumin-engineered nanoparticle formulation of paclitaxel, and doxetaxel; chloranbucil; 6-thioguanine; mercaptopurine; methotrexate; platinum analogs such as cisplatin and carboplatin; vinblastine; platinum; etoposide (VP-16); ifosfamide; mitoxantrone; vincristine; oxaliplatin; leucovovin; vinorelbine; novantrone; edatrexate; daunomycin; aminopterin; ibandronate; topoisomerase inhibitor RFS 2000; difluoromethylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine and prednisolone, or FOLFOX, an abbreviation for a treatment regimen with oxaliplatin combined with 5-FU and leucovovin.

The compounds referred to in the present description are used in therapeutically effective amounts. A “therapeutically effective amount” of a compound refers to an amount that is effective from a prophylactic or therapeutic aspect. In the case of treating a tumor or cancer, the administration of a therapeutically effective amount of a compound will contribute to the intended therapeutic effect, e.g., alleviation, amelioration, palliation, or elimination of one or more manifestations of the tumor or cancer in the patient, or any other clinical result in the course of treating a tumor or cancer patient. A therapeutic effect does not necessarily occur by administration of one dose, and may occur only after administration of a series of doses. Thus, a therapeutically effective amount may be administered in one or more administrations. Such therapeutically effective amount may vary according to factors such as the disease state, age, sex, and weight of the individual, and the ability of the compound to elicit a desired response in the individual. A therapeutically effective amount is also one in which any toxic or detrimental effects of the compound are outweighed by the therapeutically beneficial effects.

The present disclosure also relates to kits and pharmaceutically acceptable compositions comprising the above-defined topoisomerase inhibitor, ATM inhibitor and agent stimulating the immune system.

“Pharmaceutically acceptable” indicates that the substance or composition must be compatible chemically and/or toxicologically, with the other ingredients comprising a formulation, and/or the subject being treated therewith. “Pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof.

The compositions of the present disclosure may be in a variety of forms. These include, for example, liquid, semi-solid and solid dosage forms, such as liquid solutions (e.g., injectable and infusible solutions), dispersions or suspensions, tablets, pills, powders, liposomes, and suppositories. The preferred form depends on the intended mode of administration and therapeutic application. Typical preferred compositions are in the form of injectable or infusible solutions, such as compositions similar to those used for passive immunization of humans. The preferred mode of administration is parenteral (e.g., intravenous, subcutaneous, intraperitoneal, or intramuscular). In a preferred embodiment, the composition is administered by intravenous infusion or injection. In another preferred embodiment, the composition is administered by intramuscular or subcutaneous injection.

Liquid dosage forms for oral administration include, but are not limited to, pharmaceutically acceptable emulsions, microemulsions, solutions, suspensions, syrups and elixirs. In addition to the topoisomerase inhibitor, ATM inhibitor and/or agent stimulating the immune system, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, lavouring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of the topoisomerase inhibitor, ATM inhibitor and/or agent stimulating the immune system, it is often desirable to slow absorption from subcutaneous or intramuscular injection. This may be accomplished by the use of a liquid suspension of crystalline or amorphous material with poor water solubility. The rate of absorption then depends upon its rate of dissolution that, in turn, may depend upon crystal size and crystalline form. Alternatively, delayed absorption of parenterally administered topoisomerase inhibitor, ATM inhibitor and/or agent stimulating the immune system is accomplished by dissolving or suspending the compound in an oil vehicle. Injectable depot forms are made by forming microencapsule matrices of topoisomerase inhibitor, ATM inhibitor and/or agent stimulating the immune system in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories, which can be prepared by mixing the compounds of this invention with suitable non-irritating excipients or carriers such as cocoa butter, polyethylene glycol or a suppository wax, which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol and silicic acid, b) binders such as, for example, carboxymethylcellulose, alginates, gelatin, polyvinylpyrrolidinone, sucrose and acacia, c) humectants such as glycerol, d) disintegrating agents such as agar-agar, calcium carbonate, potato or tapioca starch, alginic acid, certain silicates and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hardfilled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The topoisomerase inhibitor, ATM inhibitor and/or agent stimulating the immune system can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms, the topoisomerase inhibitor, ATM inhibitor and/or agent stimulating the immune system may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of the topoisomerase inhibitor, ATM inhibitor and/or agent stimulating the immune system include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Typically, the topoisomerase inhibitor, ATM inhibitor and/or agent stimulating the immune system are incorporated into pharmaceutical compositions suitable for administration to a subject, wherein the pharmaceutical composition comprises the topoisomerase inhibitor, ATM inhibitor and/or agent stimulating the immune system and a pharmaceutically acceptable carrier. In many cases, it is preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the topoisomerase inhibitor, ATM inhibitor and/or agent stimulating the immune system.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the topoisomerase inhibitor, ATM inhibitor and/or agent stimulating the immune system in the required amount in an appropriate solvent with one or a combination of ingredients enumerated above, as required, followed by filtered sterilization. Generally, dispersions are prepared by incorporating the active ingredient into a sterile vehicle that contains a basic dispersion medium and the required other ingredients from those enumerated above. In the case of sterile powders for the preparation of sterile injectable solutions, the preferred methods of preparation are vacuum drying and freeze-drying that yield a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion, and by the use of surfactants. Prolonged absorption of injectable compositions can be brought about by including in the composition an agent that delays absorption, for example, monostearate salts and gelatin.

In one embodiment, avelumab is a sterile, clear, and colorless solution intended for IV administration. The contents of the avelumab vials are non-pyrogenic, and do not contain bacteriostatic preservatives. Avelumab is formulated as a 20 mg/mL solution and is supplied in single-use glass vials, stoppered with a rubber septum and sealed with an aluminum polypropylene flip-off seal. For administration purposes, avelumab must be diluted with 0.9% sodium chloride (normal saline solution). Tubing with in-line, low protein binding 0.2 micron filter made of polyether sulfone (PES) is used during administration.

In a further aspect, the invention relates to a kit comprising one compound selected from the group consisting of a topoisomerase inhibitor, an ATM inhibitor and an agent stimulating the immune system and a package insert comprising instructions for using any of the three foregoing compounds with the other two compounds in combination to treat a tumor, preferably a cancer, in a subject. Also provided is a kit comprising an agent stimulating the immune system, preferably an anti-PD-L1 antibody, and a package insert comprising instructions for using the agent stimulating the immune system in combination with a topoisomerase inhibitor and an ATM inhibitor to treat a tumor, preferably a cancer, in a subject. Also provided is a kit comprising a topoisomerase inhibitor, an ATM inhibitor and an agent stimulating the immune system and a package insert comprising instructions for using the topoisomerase inhibitor, ATM inhibitor and agent stimulating the immune system to treat a tumor, preferably a cancer, in a subject. The compounds of the kit may be contained in a separate container. Alternatively two or more compounds are contained in the same container. The kit can comprise a first container, a second container, a third container and a package insert, wherein the first container comprises at least one dose of a medicament comprising the topoisomerase inhibitor, the second container comprises at least one dose of a medicament comprising the ATM inhibitor, the third container comprises at least one dose of a medicament comprising the agent stimulating the immune system, and the package insert comprises instructions for treating a subject for tumor or cancer using the medicaments. The first, second and third containers may be comprised of the same or different shape (e.g., vials, syringes and bottles) and/or material (e.g., plastic or glass). The kit may further comprise other materials that may be useful in administering the medicaments, such as diluents, filters, IV bags and lines, needles and syringes.

In some embodiments, the present disclosure relates to the following:
1. A topoisomerase inhibitor, an ATM inhibitor and an agent stimulating the immune system for use in a method of treating a tumor, preferably a cancer, in a subject, wherein the topoisomerase inhibitor, the ATM inhibitor and the agent stimulating the immune system are administered simultaneously or sequentially to the subject.
2. The compounds for use according to item 1, wherein the topoisomerase inhibitor is a topoisomerase I inhibitor, preferably irinotecan.
3. The compounds for use according to item 1 or 2, wherein the ATM inhibitor is an imidazo[4,5-c]quinoline derivative, preferably Compound 1 or 8-(1,3-Dimethyl-1H-pyrazol-4-yl)-1-(3-fluoro-5-methoxy-pyridin-4-yl)-7-methoxy-3-methyl-1,3-dihydro-imidazo[4,5-c]quinolin-2-one, more preferably Compound 1.
4. The compounds for use according to any one of items 1 to 3, wherein the agent stimulating the immune system is an immune checkpoint inhibitor, preferably a PD-1 antagonist, more preferably an anti-PD-L1 antibody.
5. The compounds for use according to any one of items 1 to 4, wherein the topoisomerase inhibitor is irinotecan, the ATM inhibitor is Compound 1 and the agent stimulating the immune system is a PD-1 antagonist.
6. The compounds for use according to any one of items 1 to 5, wherein the tumor or cancer is selected from the group consisting of colorectal, breast, ovarian, pancreatic, gastric, prostate, renal, cervical, myeloma, lymphoma, leukemia, thyroid, endometrial, uterine, bladder, neuroendocrine, head and neck, liver, nasopharyngeal, testicular, small cell lung cancer, nonsmall cell lung cancer, melanoma, basal cell skin cancer, squamous cell skin cancer, dermatofibrosarcoma protuberans, Merkel cell carcinoma, glioblastoma, glioma, sarcoma, mesothelioma, and myelodisplastic syndromes.
7. The compounds for use according to any one of items 1 to 5, wherein the tumor or cancer expresses ATM.
8. A method of treating a tumor, preferably a cancer, in a subject, wherein the method comprises simultaneously or sequentially administering a topoisomerase inhibitor, an ATM inhibitor and an agent stimulating the immune system to the subject.
9. The method according to item 8, wherein the topoisomerase inhibitor is a topoisomerase I inhibitor, preferably irinotecan.
10. The method according to item 8 or 9, wherein the ATM inhibitor is an imidazo[4,5-c]quinoline derivative, preferably Compound 1 or 8-(1,3-Dimethyl-1H-pyrazol-4-yl)-1-(3-fluoro-5-methoxy-pyridin-4-yl)-7-methoxy-3-methyl-1,3-dihydro-imidazo[4,5-c]quinolin-2-one, more preferably Compound 1.
11. The method according to any one of items 8 to 10, wherein the agent stimulating the immune system is an immune checkpoint inhibitor, preferably a PD-1 antagonist, more preferably an anti-PD-L1 antibody.
12. The method according to any one of items 8 to 11, wherein the topoisomerase inhibitor is irinotecan, the ATM inhibitor is Compound 1 and the agent stimulating the immune system is a PD-1 antagonist.
13. The method according to any one of items 8 to 12, wherein the tumor or cancer is selected from the group consisting of colorectal, breast, ovarian, pancreatic, gastric, prostate, renal, cervical, myeloma, lymphoma, leukemia, thyroid, endometrial, uterine, bladder, neuroendocrine, head and neck, liver, nasopharyngeal, testicular, small cell lung cancer, nonsmall cell lung cancer, melanoma, basal cell skin cancer, squamous cell skin cancer, dermatofibrosarcoma protuberans, Merkel cell carcinoma, glioblastoma, glioma, sarcoma, mesothelioma, and myelodisplastic syndromes.
14. The method according to any one of items 8 to 13, wherein the tumor or cancer expresses ATM.
15. A kit or composition comprising:
   (a) a topoisomerase inhibitor;
   (b) an ATM inhibitor; and
   (c) an agent stimulating the immune system.
16. The kit or composition according to item 15, wherein the topoisomerase inhibitor is a topoisomerase I inhibitor, preferably irinotecan.
17. The kit or composition according to item 15 or 16, wherein the ATM inhibitor is an imidazo[4,5-c]quinoline derivative, preferably Compound 1 or 8-(1,3-Dimethyl-1H-pyrazol-4-yl)-1-(3-fluoro-5-methoxy-pyridin-4-yl)-7-methoxy-3-methyl-1,3-dihydro-imidazo[4,5-c]quinolin-2-one, more preferably Compound 1.
18. The kit or composition according to any one of items 15 to 17, wherein the agent stimulating the immune system is an immune checkpoint inhibitor, preferably a PD-1 antagonist, more preferably an anti-PD-L1 antibody.
19. The kit or composition according to any one of items 15 to 18, wherein the topoisomerase inhibitor is irinotecan, the ATM inhibitor is Compound 1 and the agent stimulating the immune system is a PD-1 antagonist.
20. The kit or composition according to any one of items 15 to 19 for use as a medicament.
21. The kit or composition according to any one of items 15 to 19 for use in a method of treating a tumor, preferably a cancer.
22. The kit or composition for use according to item 21, wherein the tumor or cancer is selected from the group consisting of colorectal, breast, ovarian, pancreatic, gastric, prostate, renal, cervical, myeloma, lymphoma, leukemia, thyroid, endometrial, uterine, bladder, neuroendocrine, head and neck, liver, nasopharyngeal, testicular, small cell lung cancer, nonsmall cell lung cancer, melanoma, basal cell skin cancer, squamous cell skin cancer, dermatofibrosarcoma protuberans, Merkel cell carcinoma, glioblastoma, glioma, sarcoma, mesothelioma, and myelodisplastic syndromes.
23. The kit or composition for use according to item 21 or 22, wherein the tumor or cancer expresses ATM.
24. A method of treating a tumor, preferably a cancer, in a subject, wherein the method comprises simultaneously or sequentially administering the composition or compounds of the kit according to any one of items 15 to 19 to the subject.
25. The method according to item 24, wherein the tumor or cancer is selected from the group consisting of colorectal, breast, ovarian, pancreatic, gastric, prostate, renal, cervical, myeloma, lymphoma, leukemia, thyroid, endometrial, uterine, bladder, neuroendocrine, head and neck, liver, nasopharyngeal, testicular, small cell lung cancer, nonsmall cell lung cancer, melanoma, basal cell skin cancer, squamous cell skin cancer, dermatofibrosarcoma protuberans, Merkel cell carcinoma, glioblastoma, glioma, sarcoma, mesothelioma, and myelodisplastic syndromes.
26. The method according to item 24 or 25, wherein the tumor or cancer expresses ATM.
27. A topoisomerase inhibitor for use in treating a tumor, preferably a cancer, by inducing immunogenic cell death.
28. The topoisomerase inhibitor for use according to item 27, wherein the tumor or cancer is selected from the group consisting of colorectal, breast, ovarian, pancreatic, gastric, prostate, renal, cervical, myeloma, lymphoma, leukemia, thyroid, endometrial, uterine, bladder, neuroendocrine, head and neck, liver, nasopharyngeal, testicular, small cell lung cancer, nonsmall cell lung cancer, melanoma, basal cell skin cancer, squamous cell skin cancer, dermatofibrosarcoma protuberans, Merkel cell carcinoma, glioblastoma, glioma, sarcoma, mesothelioma, and myelodisplastic syndromes.
29. The topoisomerase inhibitor for use according to item 27 or 28, wherein the tumor or cancer expresses ATM.
30. A topoisomerase inhibitor for use in inducing immunogenic cell death, preferably of a cell expressing ATM.
31. The topoisomerase inhibitor for use according to any one of items 27 to 30, wherein the topoisomerase inhibitor is a topoisomerase I inhibitor, preferably irinotecan.
32. The topoisomerase inhibitor for use according to any one of items 27 to 31, wherein the topoisomerase inhibitor is simultaneously or sequentially administered with an ATM inhibitor.
33. The topoisomerase inhibitor for use according to item 32, wherein the ATM inhibitor is an imidazo[4,5-c]quinoline derivative, preferably Compound 1 or 8-(1,3-Dimethyl-1H-pyrazol-4-yl)-1-(3-fluoro-5-methoxy-pyridin-4-yl)-7-methoxy-3-methyl-1,3-dihydro-imidazo[4,5-c]quinolin-2-one, more preferably Compound 1.
34. The topoisomerase inhibitor for use according to any one of items 27 to 33, wherein the topoisomerase inhibitor is simultaneously or sequentially administered with an agent stimulating the immune system.
35. The topoisomerase inhibitor for use according to item 34, wherein the agent stimulating the immune system is an immune checkpoint inhibitor, preferably a PD-1 antagonist, more preferably an anti-PD-L1 antibody.
36. The topoisomerase inhibitor for use according to any one of items 27 to 35, wherein the topoisomerase inhibitor is irinotecan, the ATM inhibitor is Compound 1 and the agent stimulating the immune system is a PD-1 antagonist.
37. A method of inducing immunogenic cell death, preferably of a cell expressing ATM, in a subject, wherein a topoisomerase inhibitor is administered to the subject.
38. The method of inducing immunogenic cell death in a subject according to item 37, wherein the topoisomerase inhibitor is a topoisomerase I inhibitor, preferably irinotecan.
39. The method of inducing immunogenic cell death in a subject according to item 37 or 38, wherein the topoisomerase inhibitor is simultaneously or sequentially administered with an ATM inhibitor.
40. The method of inducing immunogenic cell death in a subject according to any one of items 37 to 39, wherein the ATM inhibitor is an imidazo[4,5-c]quinoline derivative, preferably Compound 1 or 8-(1,3-Dimethyl-1H-pyrazol-4-yl)-1-(3-fluoro-5-methoxy-pyridin-4-yl)-7-methoxy-3-methyl-1,3-dihydro-imidazo[4,5-c]quinolin-2-one, more preferably Compound 1.
41. The method of inducing immunogenic cell death in a subject according to any one of items 37 to 40, wherein the topoisomerase inhibitor is simultaneously or sequentially administered with an agent stimulating the immune system.
42. The method of inducing immunogenic cell death in a subject according to any one of items 37 to 41, wherein the agent stimulating the immune system is an immune checkpoint inhibitor, preferably a PD-1 antagonist, more preferably an anti-PD-L1 antibody.
43. The method of inducing immunogenic cell death in a subject according to any one of items 37 to 42, wherein the topoisomerase inhibitor is irinotecan, the ATM inhibitor is Compound 1 and the agent stimulating the immune system is a PD-1 antagonist.
44. A topoisomerase inhibitor, an ATM inhibitor and an agent stimulating the immune system for use in a method of treating a tumour, preferably a cancer, by inducing immunogenic cell death in a subject, wherein the topoisomerase inhibitor, the ATM inhibitor and the agent stimulating the immune system are simultaneously or sequentially administered to the subject.
45. The compounds for use according to item 44, wherein the topoisomerase inhibitor is a topoisomerase I inhibitor, preferably irinotecan.
46. The compounds for use according to item 44 or 45, wherein the ATM inhibitor is an imidazo[4,5-c]quinoline derivative, preferably Compound 1 or 8-(1,3-Dimethyl-1H-pyrazol-4-yl)-1-(3-fluoro-5-methoxy-pyridin-4-yl)-7-methoxy-3-methyl-1,3-dihydro-imidazo[4,5-c]quinolin-2-one, more preferably Compound 1.
47. The compounds for use according to any one of items 44 to 46, wherein the agent stimulating the immune system is an immune checkpoint inhibitor, preferably a PD-1 antagonist, more preferably an anti-PD-L1 antibody.
48. The compounds for use according to any one of items 44 to 47, wherein the topoisomerase inhibitor is irinotecan, the ATM inhibitor is Compound 1 and the agent stimulating the immune system is a PD-1 antagonist.
49. The compounds for use according to any one of items 44 to 48, wherein the tumor or cancer is selected from the group consisting of colorectal, breast, ovarian, pancreatic, gastric, prostate, renal, cervical, myeloma, lymphoma, leukemia, thyroid, endometrial, uterine, bladder, neuroendocrine, head and neck, liver, nasopharyngeal, testicular, small cell lung cancer, nonsmall cell lung cancer, melanoma, basal cell skin cancer, squamous cell skin cancer, dermatofibrosarcoma protuberans, Merkel cell carcinoma, glioblastoma, glioma, sarcoma, mesothelioma, and myelodisplastic syndromes.
50. The compounds for use according to any one of items 44 to 49, wherein the tumor or cancer expresses ATM.
51. A method of treating a tumor, preferably a cancer, by inducing immunogenic cell death in a subject, wherein the method comprises simultaneously or sequentially administering a topoisomerase inhibitor, an ATM inhibitor and an agent stimulating the immune system to the subject.
52. The method according to item 51, wherein the topoisomerase inhibitor is a topoisomerase I inhibitor, preferably irinotecan.
53. The method according to item 51 or 52, wherein the ATM inhibitor is an imidazo[4,5-c]quinoline derivative, preferably Compound 1 or 8-(1,3-Dimethyl-1H-pyrazol-4-yl)-1-(3-fluoro-5-methoxy-pyridin-4-yl)-7-methoxy-3-methyl-1,3-dihydro-imidazo[4,5-c]quinolin-2-one, more preferably Compound 1.
54. The method according to any one of items 51 to 53, wherein the agent stimulating the immune system is an immune checkpoint inhibitor, preferably a PD-1 antagonist, more preferably an anti-PD-L1 antibody.
55. The method according to any one of items 51 to 54, wherein the topoisomerase inhibitor is irinotecan, the ATM inhibitor is Compound 1 and the agent stimulating the immune system is a PD-1 antagonist.
56. The method according to any one of items 51 to 55, wherein the tumor or cancer is selected from the group consisting of colorectal, breast, ovarian, pancreatic, gastric, prostate, renal, cervical, myeloma, lymphoma, leukemia, thyroid, endometrial, uterine, bladder, neuroendocrine, head and neck, liver, nasopharyngeal, testicular, small cell lung cancer, nonsmall cell lung cancer, melanoma, basal cell skin cancer, squamous cell skin cancer, dermatofibrosarcoma protuberans, Merkel cell carcinoma, glioblastoma, glioma, sarcoma, mesothelioma, and myelodisplastic syndromes.
57. The method according to any one of items 51 to 56, wherein the tumor or cancer expresses ATM.
58. Use of a topoisomerase inhibitor for the manufacture of a medicament for the treatment of a tumor, preferably a cancer, in a subject, wherein the topoisomerase inhibitor is simultaneously or sequentially administered with an ATM inhibitor and an agent stimulating the immune system to the subject.
59. The use according to item 58, wherein the topoisomerase inhibitor is a topoisomerase I inhibitor, preferably irinotecan, or a pharmaceutically acceptable salt thereof.
60. The use according to item 58 or 59, wherein the ATM inhibitor is an imidazo[4,5-c]quinoline derivative, preferably Compound 1 or 8-(1,3-Dimethyl-1H-pyrazol-4-yl)-1-(3-fluoro-5-methoxy-pyridin-4-yl)-7-methoxy-3-methyl-1,3-dihydro-imidazo[4,5-c]quinolin-2-one, more preferably Compound 1.
61. The use according to any one of items 58 to 60, wherein the agent stimulating the immune system is an immune checkpoint inhibitor, preferably a PD-1 antagonist, more preferably an anti-PD-L1 antibody.
62. The use according to any one of items 58 to 61, wherein the topoisomerase inhibitor is irinotecan, or a pharmaceutically acceptable salt thereof, the ATM inhibitor is Compound 1 and the agent stimulating the immune system is a PD-1 antagonist.
63. The use according to any one of items 58 to 62, wherein the tumor or cancer is selected from the group consisting of colorectal, breast, ovarian, pancreatic, gastric, prostate, renal, cervical, myeloma, lymphoma, leukemia, thyroid, endometrial, uterine, bladder, neuroendocrine, head and neck, liver, nasopharyngeal, testicular, small cell lung cancer, nonsmall cell lung cancer, melanoma, basal cell skin cancer, squamous cell skin cancer, dermatofibrosarcoma protuberans, Merkel cell carcinoma, glioblastoma, glioma, sarcoma, mesothelioma, and myelodisplastic syndromes.
64. The use according to any one of items 58 to 63, wherein the tumor or cancer expresses ATM.
65. A topoisomerase inhibitor, an ATM inhibitor and an agent stimulating the immune system for use as a medicament, wherein the topoisomerase inhibitor, the ATM inhibitor and the agent stimulating the immune system are administered simultaneously or sequentially to the subject.
66. The compounds for use according to item 65, wherein the topoisomerase inhibitor is a topoisomerase I inhibitor, preferably irinotecan.
67. The compounds for use according to item 65 or 66, wherein the ATM inhibitor is an imidazo[4,5-c]quinoline derivative, preferably Compound 1 or 8-(1,3-Dimethyl-1H-pyrazol-4-yl)-1-(3-fluoro-5-methoxy-pyridin-4-yl)-7-methoxy-3-methyl-1,3-dihydro-imidazo[4,5-c]quinolin-2-one, more preferably Compound 1.
68. The compounds for use according to any one of items 65 to 67, wherein the agent stimulating the immune system is an immune checkpoint inhibitor, preferably a PD-1 antagonist, more preferably an anti-PD-L1 antibody.
69. The compounds for use according to any one of items 65 to 68, wherein the topoisomerase inhibitor is irinotecan, the ATM inhibitor is Compound 1 and the agent stimulating the immune system is a PD-1 antagonist.

All the references cited herein are incorporated by reference in the disclosure of the invention hereby.

It is to be understood that this invention is not limited to the particular molecules, pharmaceutical compositions, uses and methods described herein, as such matter can, of course, vary. It is also to be understood that the terminology used herein is for the purpose of describing particular embodiments only and is not intended to limit the scope of the present invention, which is only defined by the appended claims. The techniques that are essential according to the invention are described in detail in the specification. Other techniques which are not described in detail correspond to known standard methods that are well known to a person skilled in the art, or the techniques are described in more detail in cited references, patent applications or standard literature. Provided that no other hints in the application are given, they are used as examples only, they are not considered to be essential according to the invention, but they can be replaced by other suitable tools and biological materials. Furthermore, each of the embodiments described herein envisions within its scope pharmaceutically acceptable salts of the compounds described herein. Accordingly, the phrase “or a pharmaceutically acceptable salt thereof” is implicit in the description of all compounds described herein.

Any features, including optional, suitable, and preferred features, described in relation to any particular aspect of the invention may also be features, including optional, suitable and preferred features, of any other aspect of the present invention.

Although methods and materials similar or equivalent to those described herein can be used in the practice or testing of the present invention, suitable examples are described below. Within the examples, standard reagents and buffers that are free from contaminating activities (whenever practical) are used. The examples are particularly to be construed such that they are not limited to the explicitly demonstrated combinations of features, but the exemplified features may be unrestrictedly combined again provided that the technical problem of the invention is solved. Similarly, the features of any claim can be combined with the features of one or more other claims. The present invention having been described in summary and in detail, is illustrated and not limited by the following examples.

### EXAMPLES

### Example 1

### Assessing the Effect of ATM Inhibitors in Combination With Topoisomerase Inhibitors on the Growth of 35 Cell Lines

Two ATM inhibitors (imidazo[4,5-c]quinoline derivatives, wherein “ATMi 1” corresponds to Compound 1) were independently tested in combination with various chemotherapeutic agents, including the topoisomerase I inhibitors SN-38 (the active metabolite of irinotecan) and topotecan and the topoisomerase II inhibitor etoposide, to analyze the combinatorial effect on cell growth inhibition. The ATM inhibitors have been used at 1 μM and the topoisomerase inhibitors have been used in increasing concentrations: SN38 at 3.91E-10M, 1.56E-09M, 6.25E-09M, 2.5E-08M, and 1E-07M, topotecan at 1.95E-09M, 7.81E-09M, 3.13E-08M, 1.25E-07M, and 5E-07M, and etoposide at 1.95E-08M, 7.810E-08M, 3.13E-07M, 1,25E-06M, and 5E-06M. 35 different cancer cell lines were treated with said combinations.

Experimental conditions/treatment schedule: Cells were seeded in 96-well microtiter plates under standard conditions. The cells were allowed to stand for 48 hours prior to treatment. The treatment was performed for 120 hours and stopped by addition of trichloracetic acid followed by Sulforhodamine B staining. Combinations included simultaneous addition of both agent pairs.

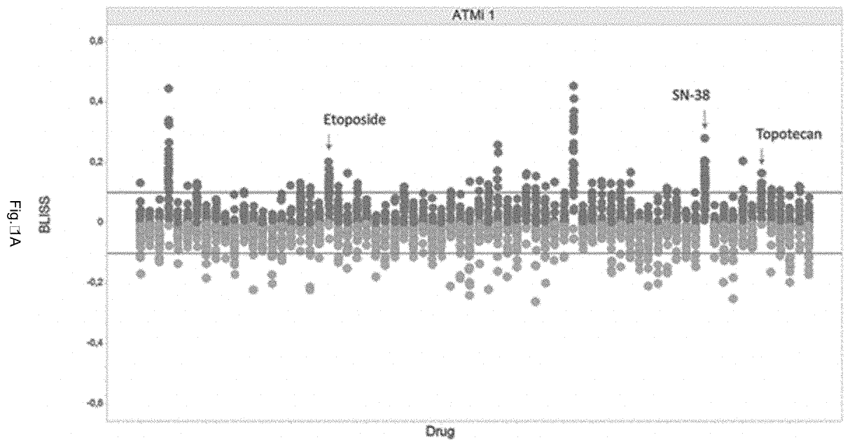

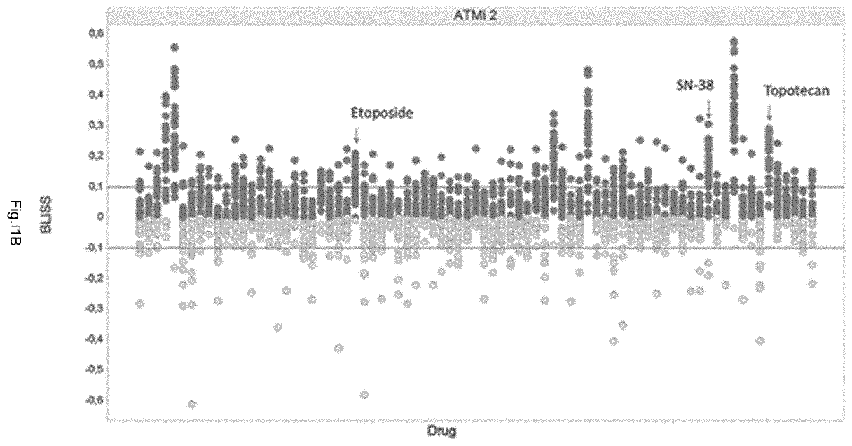

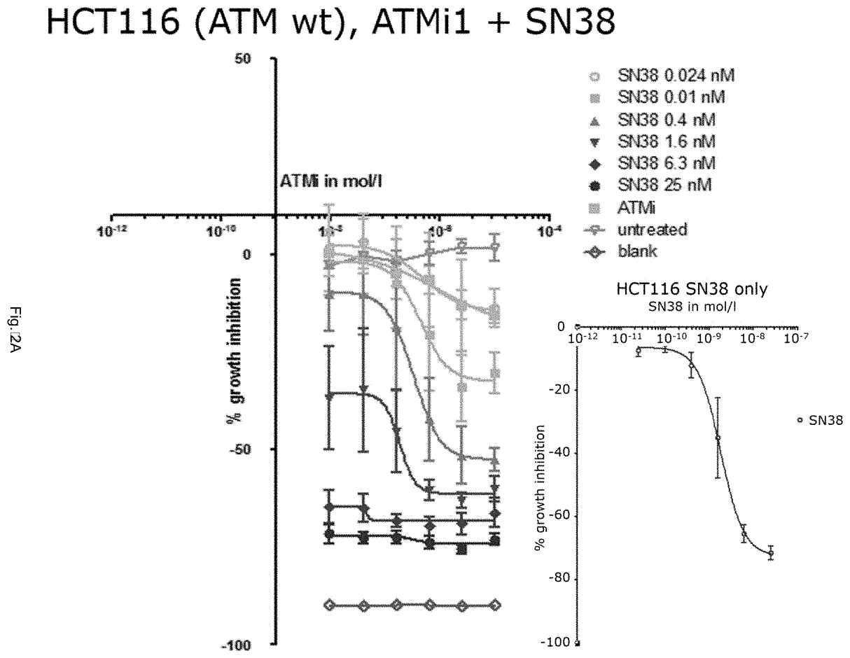

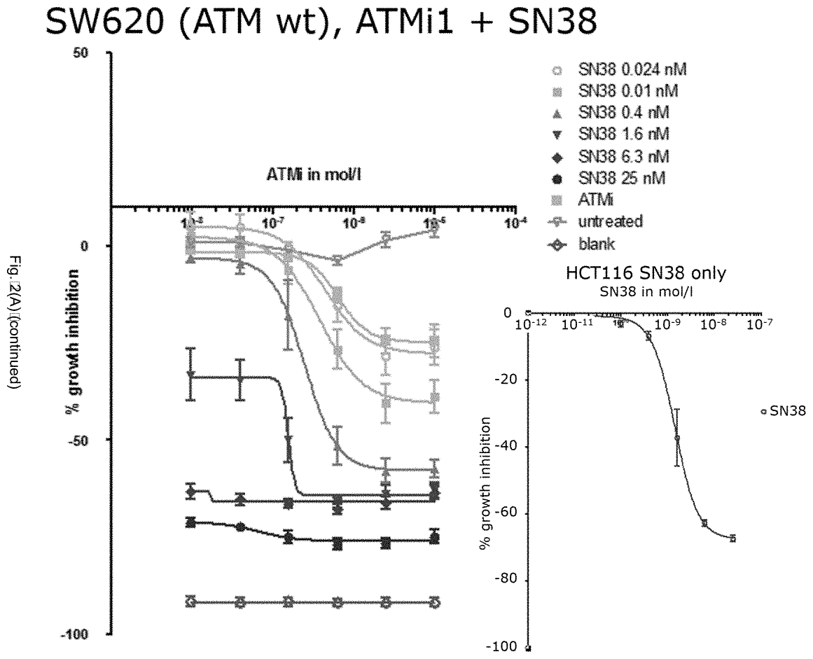

The combinatorial effect of the compounds was determined by measuring their cell growth inhibition as compared to the inhibition observed for monotherapies of these compounds using the same concentrations as used for the combinations. The combination effect has been calculated as the excess over the linear combination of the monotherapy effects using the BLISS independence model (E1+2=E1+E2−E1 E2). The average BLISS excess is calculated as the average excess over the linear combination of the monotherapy effects across all inhibitor concentrations. Positive BLISS excess values above 0.1 describe a synergistic effect, and BLISS excess values below −0.1 describe an antagonistic effect. BLISS excess measures of the combination of two ATM inhibitors and various chemotherapeutic agents, including the mentioned topoisomerase inhibitors, for 35 cell lines are shown in FIG. 1.

### Example 2

### Assessing the Effect of ATM Inhibitors in Combination With Topoisomerase Inhibitors on Cell Growth in vitro

Two ATMi inhibitors (imidazo[4,5-c]quinoline derivatives, wherein “ATMi 1” corresponds to Compound 1) were tested in combination with the active metabolite of irinotecan (SN38). The inhibitors for ATM and SN38 were tested in a serial dilution in a matrix setup, so that each concentration of ATM inhibitors is tested in combination with each concentration of the combination partner. 2.5E-08M, 6.3E-09M,1.6E-09M, 4E-10M, 9.8E-11, 2.4E-11M and 0M was used for SN38 and 1.0E-5M, 2.5E-06M, 6.3E-07, 1.6E-07, 3.9E-08, 9.8E-09M and 0M for the ATM inhibitors. Cells were seeded on 96 well plates and on the next day the compounds were added. After 72 h viability was measured by using Alamar Blue reagent. Normalization was done by setting the blank control to −100% and the untreated control to 0%.

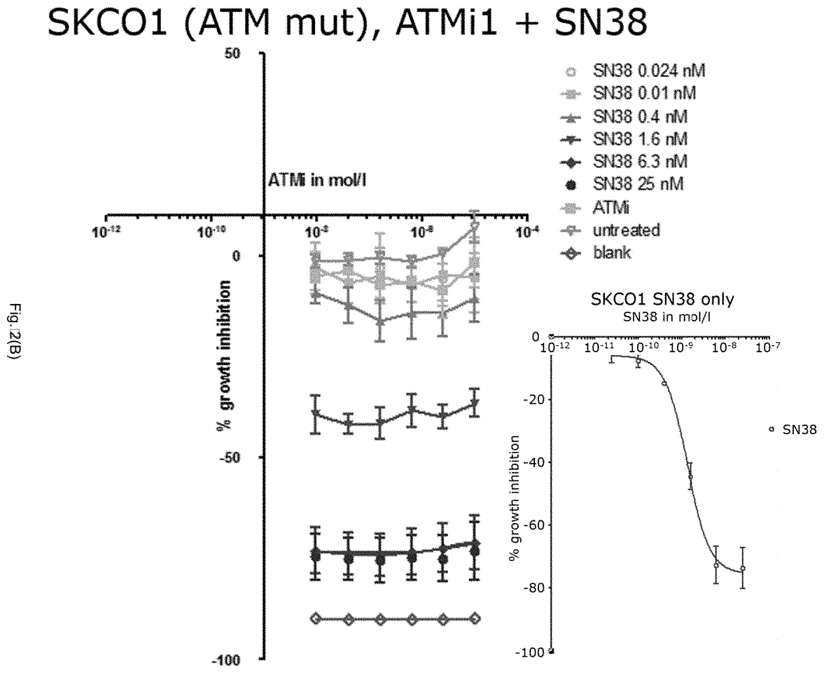

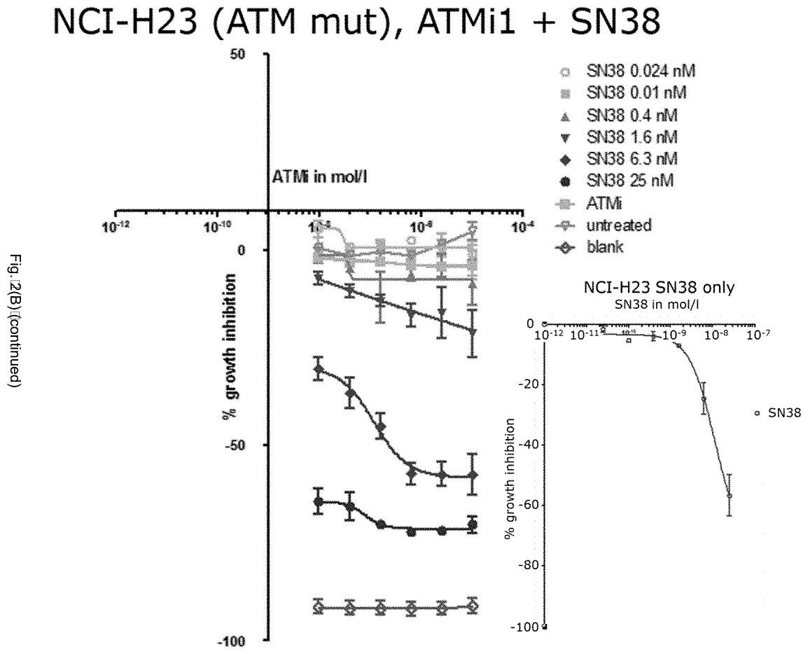

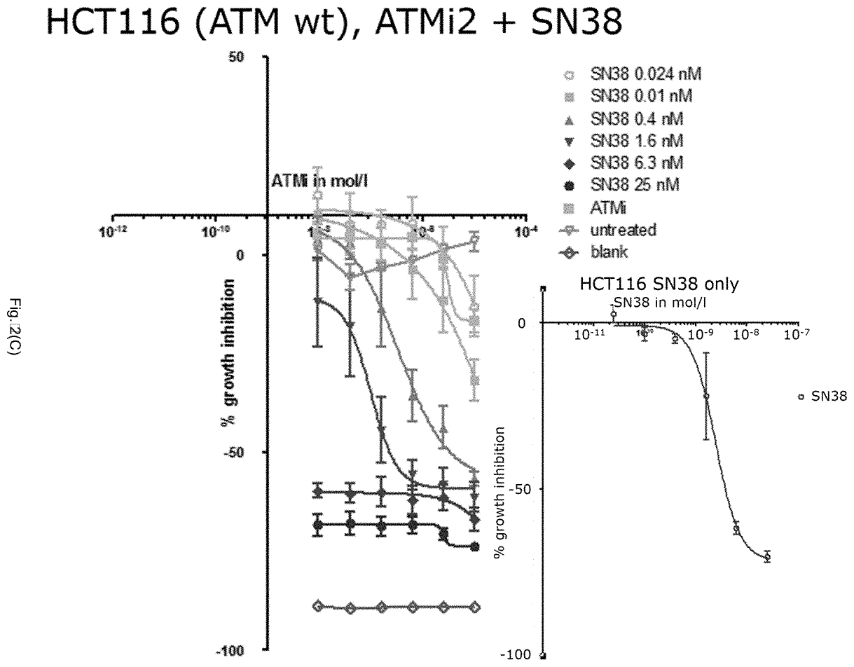

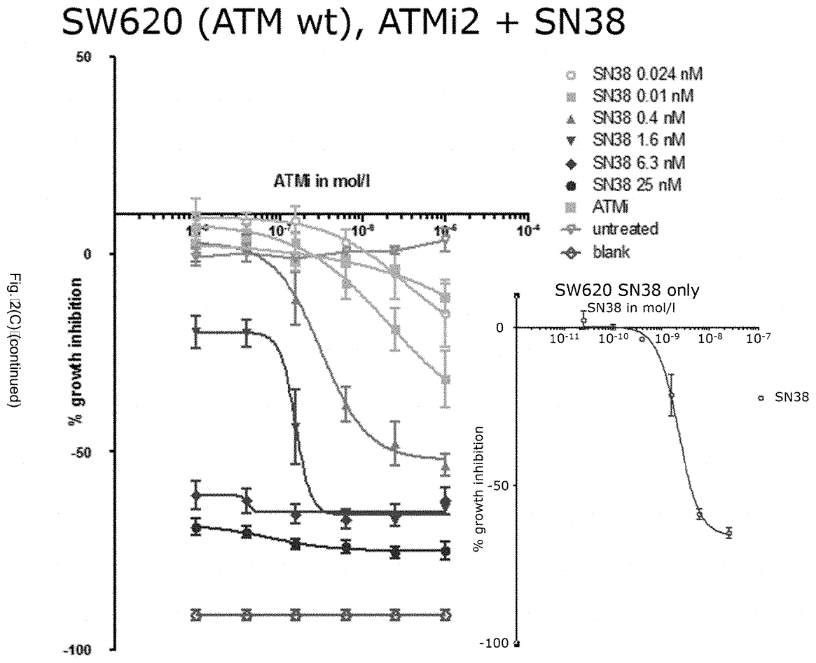

Growth inhibition is shown in FIG. 2 for each ATMi concentration in presence of a fixed concentration of SN38. For comparison the growth inhibition induced by a serial dilution of SN38 is also indicated. A clear combination effect leading to a shift of the inflection point of the concentration response curves to lower ATMi concentrations and to an increased growth inhibition could be demonstrated on cell lines expressing a wildtype ATM (colon carcinoma cell lines HCT116 and SW620) by combining with SN38. For cell lines expressing no ATM (SKCO1 and NCI-H23) no combination effect could be detected. In this case the induced growth inhibition is only a consequence of the topoisomerase inhibition. The effect could be demonstrated with the two different ATMi.

### Example 3

### Assessing the Therapeutic Effect of ATM Inhibitors in Combination With Topoisomerase Inhibitors in a CRC Xenograft Model

The therapeutic effect of two different ATM inhibitors (imidazo[4,5-c]quinoline derivatives, wherein “ATMi 1” corresponds to Compound 1) in combination with irinotecan were tested in the human colon cancer xenograft model SW620 in mice (subcutaneous tumor implantation). Animals were treated with three 1-week cycles of a combination of irinotecan and the respective ATM inhibitors, or the respective monotherapy control arms. One cycle consisted of one irinotecan application (ip) at a dose of 50 mg/kg, followed by 100, and 50 mg/kg of ATMi 1, or 10 and 25 mg/kg of ATMi 2 (po) 24 h later and for four subsequent days.

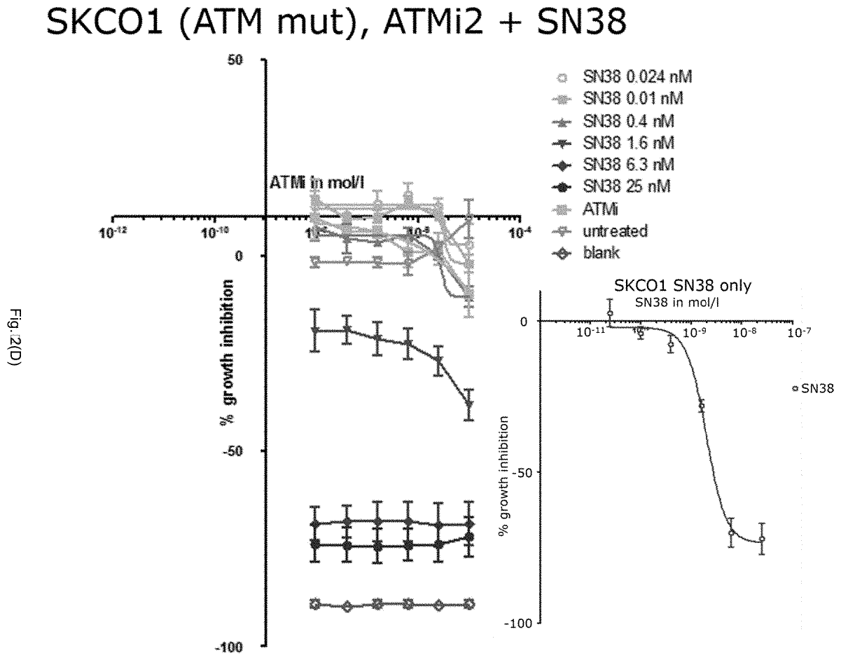

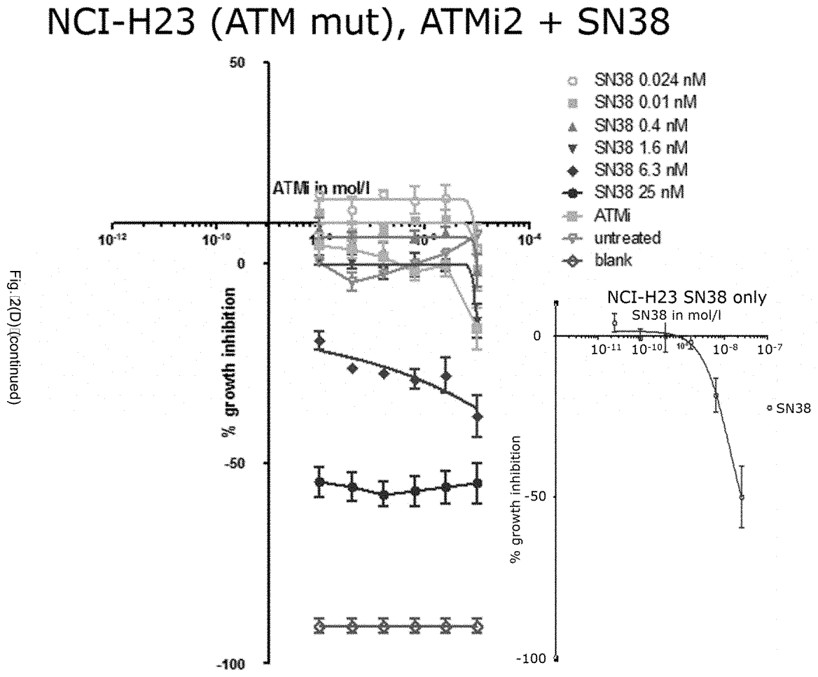

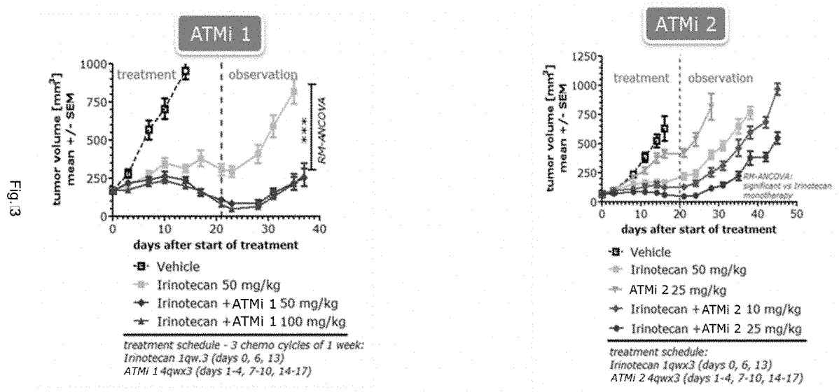

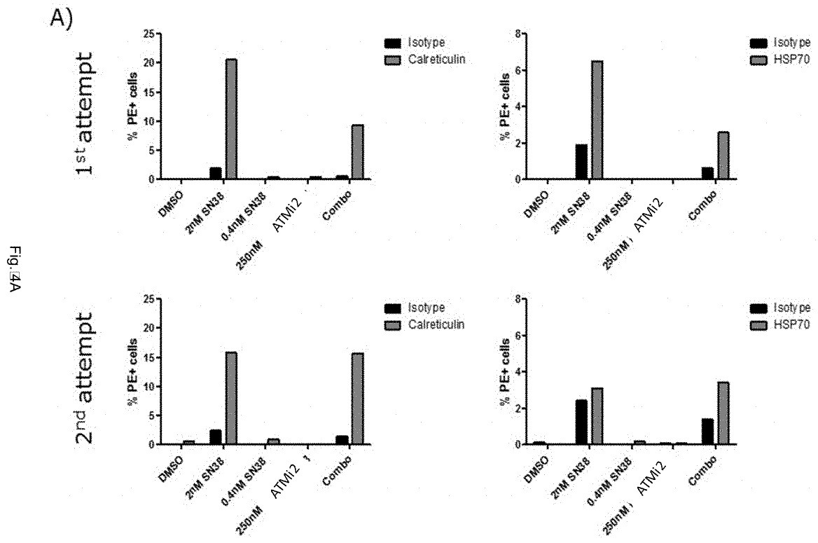

The effect of the treatments on tumor volume is shown in FIG. 3.

The addition of the respective higher ATMi doses to irinotecan resulted in significantly higher efficacy as compared to the irinotecan treatment arm. For example, in the study performed with ATMi 2 at the end of the three 1-week treatment cycles (day 21) the mean tumor volume of the respective (25 mg/kg) combination group (n=10) was 53 mm³—a decrease of ˜87% compared to starting volume, whereas the mean tumor volume of the irinotecan group (n=10) increased by 223% to ˜220 mm³. One week after stop of treatment (d28), 5/10 animals in the combination arm were, compared to tumor starting volume, still progression free (<73% increase in tumor volume) with 3/10 stable diseases (tumor volume change between 30% decrease and 73% increase) and 2/10 partial responses (tumor volume >30% decrease). In the irinotecan arm all animals showed progressive disease at day 28.

The treatment was well tolerated. No body weight loss was observed in monotherapy or combination treatment arms compared to vehicle treated animals. Both ATM inhibitors showed similar results.

### Example 4

### Assessing the Effect of ATM Inhibitors in Combination With Topoisomerase Inhibitors on Immunogenic Cell Death of a Colon Cancer Cell Line

An ATMi inhibitor has been used in combination with the active metabolite of irinotecan (SN38). The inhibitor for ATM and SN38 were tested as single agents and in combination at the concentrations of the GI50 of the combination. The GI50 of SN38 was included as a positive control for induction of immunogenic cell death. Concentrations used as single agents or in combination were 0.4 nM SN38 and 250 nM ATMi2 and the GI50 of SN38 was 2 nM. Cell surface expression of calreticulin, HSP70 and HSP90 was analysed by Flow Cytometry, ATP secretion by ATPlite assay Perkin Elmer, HMGB1 release by HMGB1 Elisa (IBL International) and apoptosis by Caspase-Glo 3/7 Assay Systems (measures caspase-3/7 activities).

Analyses of DAMPs (Calreticulin, HSP70, HSP90, HMGB1 and ATP) were carried out whithin the same sample. Cells were treated 24 hours after seeding and analyses performed 72 hours post-treatment. Cells were used for FACS analyses and supenatants for ATP and HMGB1 measurements. Caspase-Glo 3/7 was carried out in parallel following the same treatment schedule.

For FACS analyses, an unstained sample was used as a gating control and an isotype sample to measure unspecific antibody binding. % PE positive cells represent cells with cell surface expression of the tested marker (all markers were conjugated with PE and tested independently). ATP secretion, HMGB1 release and apoptosis were carried out accordingly to the protocols provided by the commercial supplier (data represented as the average of two independent biological repeats).

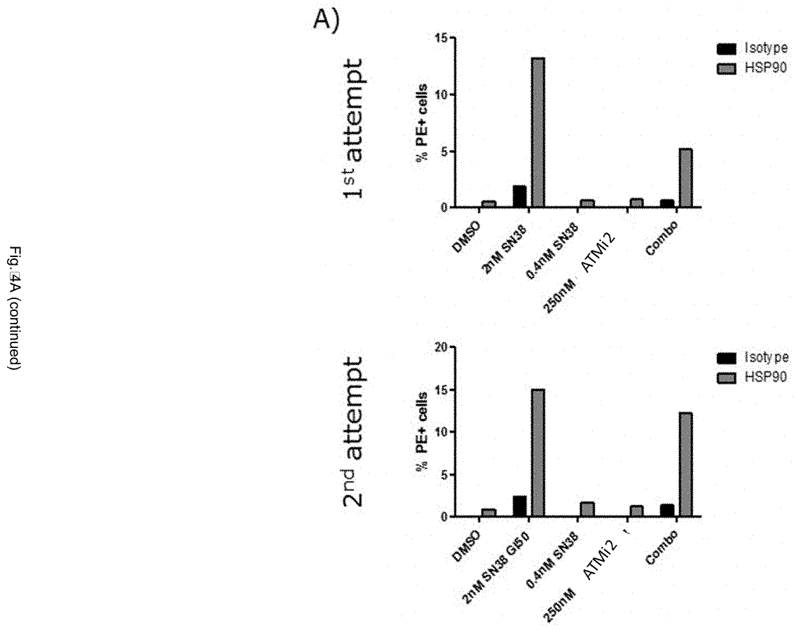

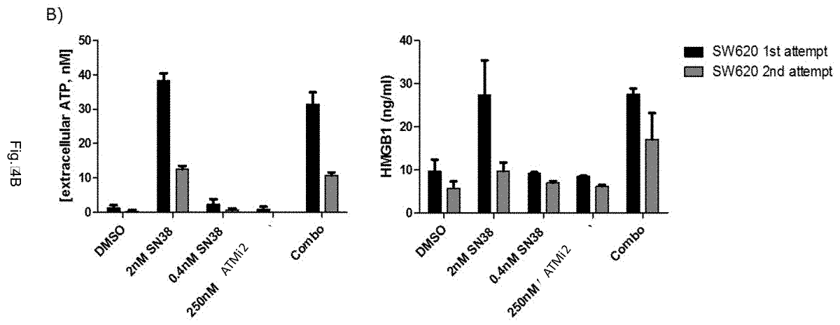

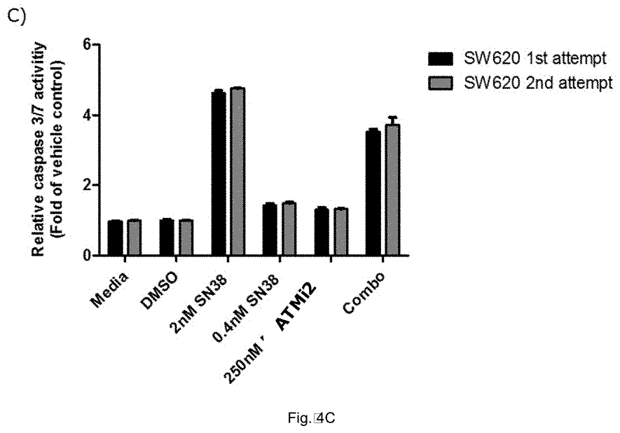

As reflected by FIG. 4, a clear induction of immunogenic cell death was observed by the increase in cell surface expression of calreticulin, Hsp70 and Hsp90, increase in ATP and HMGB1 release and apoptosis (activation of caspase-3-7) with both SN38 (G150) and the combination of ATMi2 and SN38 but not when these compounds where used as single agents at the concentrations used for the combination.

1. A method of treating a tumor in a subject comprising simultaneously or subsequentially administrating a topoisomerase inhibitor, an ataxia-telangiectasia mutated (ATM) inhibitor and an immune checkpoint inhibitor to said subject.

2. The method according to claim 1, wherein the topoisomerase inhibitor is a topoisomerase I inhibitor.

3. The method according to claim 1, wherein the ataxia-telangiectasia mutated (ATM) inhibitor is an imidazo[4,5-c]quinoline derivative or a pharmaceutically acceptable salt thereof

4. The method according to claim 1, wherein the immune checkpoint inhibitor is a PD-1 antagonist.

5. The according to claim 1, wherein the topoisomerase inhibitor is irinotecan, or a pharmaceutically acceptable salt thereof, the ATM inhibitor is 3-Fluoro-4-[7-methoxy-3-methyl-8-(1-methyl-1H-pyrazol-4-yl)-2-oxo-2,3-dihydroimidazo[4,5-c]-quinolin-1-yl]benzonitrile, or a pharmaceutically acceptable salt thereof, and the immune checkpoint inhibitor is a PD-1 antagonist.

6. The method according to claim 1, wherein the tumor is a cancer selected from the group consisting of colorectal, breast, ovarian, pancreatic, gastric, prostate, renal, cervical, myeloma, lymphoma, leukemia, thyroid, endometrial, uterine, bladder, neuroendocrine, head and neck, liver, nasopharyngeal, testicular, small cell lung cancer, nonsmall cell lung cancer, melanoma, basal cell skin cancer, squamous cell skin cancer, dermatofibrosarcoma protuberans, Merkel cell carcinoma, glioblastoma, glioma, sarcoma, mesothelioma, and myelodisplastic syndromes.

7. The method according to claim 1, wherein the tumor expresses ataxia-telangiectasia mutated (ATM).

8.-15. (canceled)

16. The method according to claim 2, wherein the topoisomerase I inhibitor is irinotecan, or a pharmaceutically acceptable salt thereof.

17. The method according to claim 1, wherein the ataxia-telangiectasia mutated (ATM) inhibitor is 3-Fluoro-4-[7-methoxy-3-methyl-8-(1-methyl-1H-pyrazol-4-yl)-2-oxo-2,3-dihydroimidazo[4,5-c]-quinolin-1-yl]benzonitrile, or a pharmaceutically acceptable salt thereof.

18. The method according to claim 5, wherein the PD-1 antagonist is an anti-PD-L1 antibody.
